(19) United States
(12) Patent Application Publication
(10) Pub. No.: US 2017/0196730 A1
(43) Pub. Date: Jul. 13, 2017

(54) Far-infrared (FIR) light heated ceramic electric blower for physiotherapeutic or other therapeutic uses

(71) Applicant: Amaxy, Inc., La Jolla, CA (US)

(72) Inventor: Shirong Chen, Wenling (CN)

(21) Appl. No.: 15/056,906

(22) Filed: Feb. 29, 2016

(30) Foreign Application Priority Data
Jan. 8, 2016 (CN) 201620010735.7

(51) Int. Cl. A61F 7/00; F04D 29/58; F04D 29/28; F04D 29/42; F04D 25/06; F04D 27/00

(52) U.S. Cl. CPC A61F 7/0085; F04D 25/06; F04D 27/006; F04D 29/281; F04D 29/4206; F04D 29/582; A61F 2007/0087; A61F 2007/0088

(57) ABSTRACT

An FIR light heated ceramic electric blower for physiotherapeutic use that belongs to the technical field of electric blowers comprises a housing and fan blades. The housing has an air inlet and an air outlet. Between the fan blades and the air outlet there is a heating element, which is an FIR heating light tube. The FIR heating light tube is accommodated within the inner chamber of the housing with a mica bracket. The circuit of the FIR heating light tube is connected with a hot air switch. This electric blower heats by having its FIR heating light tube electrified, and all of the heat is pushed to the air outlet by the rotating fan blades driven by an electric motor. The infrared rays emitted from the FIR heating light tube can quickly and effectively relieve the soreness and discomfort in joints and muscles, thus achieving the result of relieving pains in muscles and joints. This electric blower does not produce open fire while heating and has such advantages as safeness and low energy consumption.

### CROSS-REFERENCE TO RELATED APPLICATIONS

This application claims the priority and benefit of Chinese patent application ser. no. 201620010735.7 filed Jan. 8, 2016.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND AN INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

Field of Invention

This utility model belongs to the technical field of electric blowers, and specifically relates to a far-infrared (FIR) light heated ceramic electric blower for physiotherapeutic use.

Background of the Invention

The electric blower has been used for more than a century for drying hair and other items. At present, a common electric blower mainly consists of a housing, an electric motor, fan blades, a heating element, an insulation bracket, a power switch and a power cord, wherein the heating element is typically made of iron nickel chromium wires. Usually, the iron nickel chromium wires are spirally wind around the insulation bracket. According to the physics of the spiral law for conductors, such an iron nickel chromium wire structure generates a large amount of electromagnetic waves. However, the iron nickel chromium wire has some weaknesses, such as low thermal efficiency, high energy consumption and easy breaking. Under the macro environment where the energy-conserving and eco-friendly low-carbon lifestyle is promoted, it is very important to manufacture an electric blower that is energy saving, health caring and environment protecting.

### SUMMARY OF THE INVENTION

This utility model is intended to overcome the abovementioned defects and imperfections.

This utility model adopts the following technical solution to achieve its purpose:

A far-infrared (FIR) light heated ceramic electric blower for physiotherapeutic use comprises a housing and fan blades. In the housing there is an air inlet and an air outlet. Between the fan blades and the air outlet there is a heating element, which is an FIR heating light tube. The FIR heating light tube is accommodated within the inner chamber of the housing with a mica bracket. The circuit of the FIR heating light tube is connected with a hot air switch.

The housing consists of a front cylinder, a rear cylinder and a rear cover. The front cylinder and the rear cylinder are snap-fit together to form an inner chamber. There is an air outlet at the top end of the front cylinder. The rear cover is connected to the tail of the rear cylinder and has an air inlet.

The FIR light heated ceramic electric blower for physiotherapeutic use also comprises fitting strips, an air outlet ring and a hollow inner cylinder. The inner cylinder is engaged and disposed within the inner chamber of the housing, and the top end of the inner cylinder is spaced with the inner wall of the front cylinder. The air outlet ring is mounted at the top end of the inner cylinder and an air outlet ring has a set of parallel spaced deflectors. The fitting strips are engaged and disposed between the top end of the inner cylinder and the top end of the front cylinder.

The FIR light heated ceramic electric blower for physiotherapeutic use also comprises honeycomb ceramics and a step-down heating coil. The FIR heating light tube and the step-down heating coil are both fixed to the mica bracket. The FIR heating light tube and the step-down heating coil are in circuit connection. The step-down heating coil and the hot air switch are in circuit connection.

The mica bracket consists of a set of radially disposed panels, which are connected with each other on the axis of the mica bracket.

The FIR light heated ceramic electric blower for physiotherapeutic use also comprises a rubber gasket, an electric motor and an electric motor bracket. The electric motor is fixed to the inner wall of the housing with the electric motor bracket. The electric motor is connected with fan blades on one end and connected with the rubber gasket on the other end. The FIR light heated ceramic electric blower for physiotherapeutic use also comprises a cold air button, a cold air switch and a power switch. The housing has a handle, on which the cold air button, the cold air switch and the power switch are mounted, and the cold air button and the cold air switch are in circuit connection.

This electric blower has the following advantages: 1. The FIR light tube of this electric blower is electrified to heat, and all of the heat is blown to the air outlet under the pressure from the rotating fan blades driven by an electric motor. The FIR ray generated by the FIR light tube and ceramics has the shortest wavelength and the strongest penetration, thus effectively helping to relieve soreness and pain. Unlike ultraviolet ray, infrared ray does not burn the skin. The infrared light can be used for INFRACARE for one time or many times every day according to personal needs and purpose. Adopting the high-power halogen light tube technology, the FIR heating light tube can quickly and effectively achieve the result of relieving the soreness and discomfort in the joints and muscles. 2. Honeycomb ceramics are disposed between the FIR heating light tube and the air outlet. On one hand, the holes evenly distributed on the honeycomb ceramics are similar to those on the honeycomb and ensure even distribution of the heat. On the other hand, the honeycomb ceramics begins to heat and send out infrared rays after absorbing the heat from the infrared heating light tube to perform health care physiotherapy on human body. 3. The mica bracket consists of a set of radially disposed panels, which are connected with each other on the axis of the mica bracket, so that the air can go through smoothly in the axial direction with minimum air resistance and the path for air heating is extended for a high heat utilization rate. This electric blower is an ideal choice as it does not need any open fire while heating and it also has other advantages such as safety, low energy consumption and ease of operation.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is the perspective view of the structure of this utility model; and,

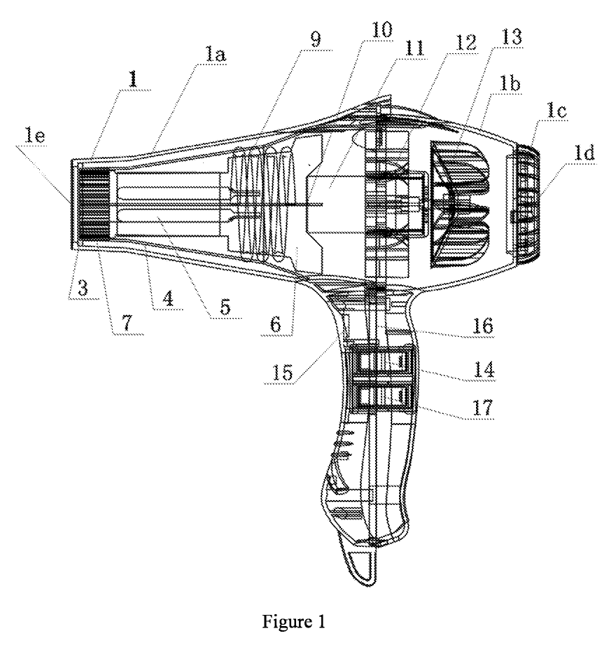

FIG. 2 is the exploded view of this utility model.

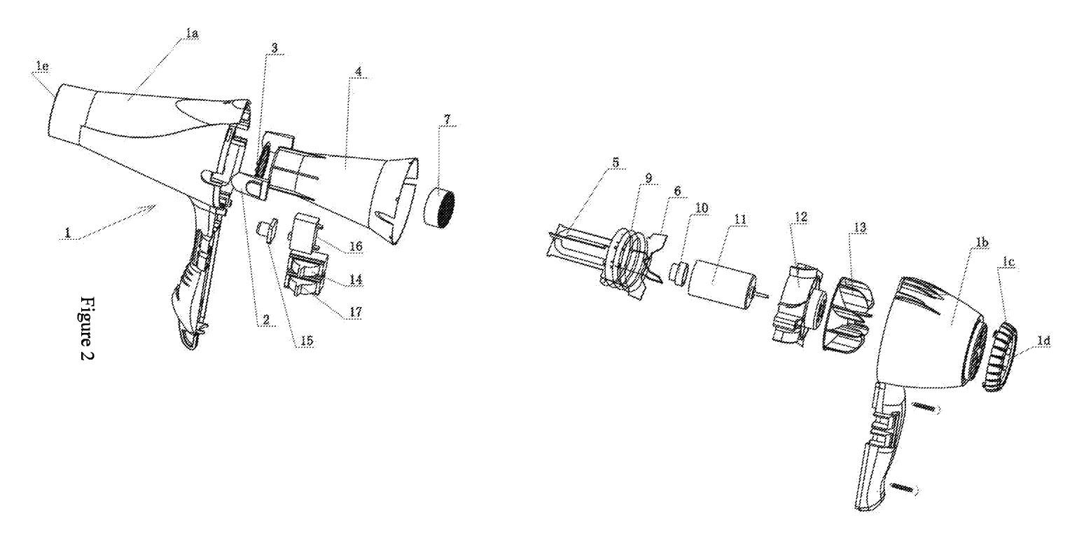

### IN THE DRAWINGS

1—housing;
   1a—front cylinder;
   1b—rear cylinder;
   1c—rear cover;
   1d—air inlet;
   1e—air outlet;
2—fitting strips;
3—air outlet ring;
4—inner cylinder;
5—FIR heating light tube;
6—mica bracket;
7—honeycomb ceramics
9—step-down heating coil;
10—rubber gasket;
11—electric motor;
12—electric motor bracket;
13—fan blades;
14—hot air switch;
15—cold air button;
16—cold air switch; and,
17—power switch.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

Below is further detailed description of this utility model by reference to the drawings.

An FIR light heated ceramic electric blower for physiotherapeutic use consists of a housing 1, fitting strips 2, an air outlet ring 3, an inner cylinder 4, an FIR heating light tube 5, a mica bracket 6, honeycomb ceramics 7, a step-down heating coil 9, a rubber gasket 10, an electric motor 11, an electric motor bracket 12, fan blades 13, a hot air switch 14, a cold air button 15, a cold air switch 16 and a power switch 17.

The housing 1 consists of a front cylinder 1a, a rear cylinder 1b and a rear cover 1c. The front cylinder 1a and the rear cylinder 1b are snap-fit together to form an inner chamber. There is an air outlet 1e at the top end of the front cylinder 1a. The rear cover 1c is connected to the tail of the rear cylinder 1b and has an air inlet 1d.

The inner cylinder 4 is engaged and disposed within the inner chamber of the housing 1, and the top end of the inner cylinder 4 is spaced with the inner wall of the front cylinder 1a. The air outlet ring 3 is mounted at the top end of the inner cylinder 4 and an air outlet ring 3 has a set of parallel spaced deflectors. The fitting strips 2 are engaged and disposed between the top end of the inner cylinder 4 and the top end of the front cylinder 1a.

Between the fan blades 13 and the air outlet 1 e there is a heating element, which is an FIR heating light tube 5. The FIR heating light tube 5 is accommodated within the inner chamber of the housing 1 with a mica bracket 6. The FIR heating light tube 5 and the step-down heating coil 9 are both fixed to the mica bracket 6. The FIR heating light tube 5 and the step-down heating coil 9 are in circuit connection. The step-down heating coil 9 and the hot air switch 14 are in circuit connection. The circuit of the FIR heating light tube 5 is connected with a hot air switch 14.

As a preferred embodiment, the mica bracket 6 consists of a set of radially disposed panels. Hence, while the mica bracket 6 holds the FIR heating light tube 5 and the step-down heating coil 9 together, the air resistance is small, which is good for ventilation.

The electric motor 11 is fixed to the inner wall of the housing 1 with the electric motor bracket 12. The electric motor 11 is connected with fan blades 13 on one end and connected with the rubber gasket 10 on the other end. The rubber gasket 10 is disposed between the electric motor 11 and the mica bracket 6 to buff the vibration of the electric motor 11.

The housing 1 has a handle, on which the cold air button 15, the cold air switch 16 and the power switch 17 are mounted, and the cold air button 15 and the cold air switch 16 are in circuit connection.

This utility model is described by reference to the preferred embodiment. Without deviating from these principles, this device may also be transformed and improved in several ways. It should be pointed out that any alternative or equivalent technical solutions to this solution shall all be covered in the scope of protection by this utility model.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such .as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

### PAPER “SEQUENCE LISTING”

Not applicable.

What is claimed is:

1. A far-infrared (FIR) light heated ceramic electric blower for physiotherapeutic use comprising:
a housing (1) and fan blades (13);
wherein the housing (1) there is an air inlet (1d) and an air outlet (1e);
wherein between the fan blades (13) and the air outlet (1e) there is a heating element, which is an FIR heating light tube (5);
wherein the FIR heating light tube (5) is accommodated within the inner chamber of the housing (1) with a mica bracket (6); and,
wherein the circuit of the FIR heating light tube (5) is connected to a hot air switch (14).

2. The FIR light heated ceramic electric blower for physiotherapeutic use according to claim 1, wherein:
the housing (1) consists of a front cylinder (1a), a rear cylinder (1b) and a rear cover (1c);
the front cylinder (1a) and the rear cylinder (1b) are snap-fit together to form an inner chamber;
there is an air outlet (1e) at the top end of the front cylinder (1a); and,
the rear cover (1c) is connected to the tail of the rear cylinder (1b) and has an air inlet (1d).

3. The FIR light heated ceramic electric blower for physiotherapeutic use according to claim 1 further comprising:
fitting strips (2);
an air outlet ring (3);
a hollow inner cylinder (4);
wherein the inner cylinder (4) is engaged and disposed within the inner chamber of the housing (1), and the top end of the inner cylinder (4) is spaced with the inner wall of the front cylinder (1a);
wherein the air outlet ring (3) is mounted at the top end of the inner cylinder (4) and an air outlet ring (3) has a set of parallel and spaced deflectors; and,
wherein the fitting strips (2) are engaged and disposed between the top end of the inner cylinder (4) and the top end of the front cylinder (1a).

4. The FIR light heated ceramic electric blower for physiotherapeutic use according to claim 1 further comprising:
honeycomb ceramics (7) and a stepdown heating coil (9);
wherein the FIR heating light tube (5) and the step-down heating coil (9) are both fixed to the mica bracket (6);
wherein the FIR heating light tube (5) and the step-down heating coil (9) are in circuit connection; and,
wherein the step-down heating coil (9) and the hot air switch (14) are in circuit connection.

5. The FIR light heated ceramic electric blower for physiotherapeutic use according to claim 4, wherein the mica bracket (6) consists of a set of radially disposed panels.

6. The FIR light heated ceramic electric blower for physiotherapeutic use according

1. m 1 further comprising:
a rubber gasket (10);
an electric motor (11);
an electric motor bracket (12);
wherein the electric motor (11) is fixed to the inner wall of the housing (1) with the electric motor bracket (12); and,
wherein the electric motor (11) is connected with fan blades (13) on one end and connected with the rubber gasket (10) on the other end.

7. The FIR light heated ceramic electric blower for physiotherapeutic use according to claim 1 further comprising:
a cold air button (15);
a cold air switch (16);
a power switch (17); and,
wherein the housing (1) has a handle, on which the cold air button (15), the cold air switch (16) and the power switch (17) are mounted, and the cold air button (15) and the cold air switch (16) are in circuit connection.
